(19) United States
(12) Patent Application Publication
(10) Pub. No.: US 2024/0257902 A1
(43) Pub. Date: Aug. 1, 2024

(54) ANTIGEN PREDICTION METHOD AND APPARATUS, DEVICE, AND STORAGE MEDIUM

(71) Applicant: TENCENT TECHNOLOGY (SHENZHEN) COMPANY LIMITED, Shenzhen (CN)

(72) Inventors: Yu ZHAO, Shenzhen (CN); Bing HE, Shenzhen (CN); Jianhua YAO, Shenzhen (CN); Xiaona SU, Shenzhen (CN); Zhimeng XU, Shenzhen (CN)

(73) Assignee: TENCENT TECHNOLOGY (SHENZHEN) COMPANY LIMITED, Shenzhen (CN)

(21) Appl. No.: 18/603,739

(22) Filed: Mar. 13, 2024

Related U.S. Application Data
(63) Continuation of application No. PCT/CN23/91052, filed on Apr. 27, 2023.

(30) Foreign Application Priority Data
Jul. 8, 2022 (CN) 202210804792.2

(51) Int. Cl. G16B 15/00; G06N 5/022; G16B 30/00; G16B 40/20

(52) U.S. Cl. CPC G16B 15/00; G06N 5/022; G16B 30/00; G16B 40/20

(57) ABSTRACT

An antigen prediction method including inputting genetic information, sequence information, and three-dimensional structure features of an immune cell receptor into an antigen prediction model, performing, by the antigen prediction model, feature extraction on the genetic information and the sequence information to obtain genetic features and sequence features of the immune cell receptor, integrating, by the antigen prediction model, the genetic features, the sequence features, and the three-dimensional structure features to obtain receptor features of the immune cell receptor, performing, by the antigen prediction model, full connection and normalization on the receptor features to output a probability of the immune cell receptor being associated with each candidate antigen of a plurality of candidate antigens, and determining, based on the probability of the immune cell receptor being associated with each candidate of the plurality of candidate antigens, an antigen binding to the immune cell receptor from the plurality of candidate antigens.

### CROSS REFERENCE TO RELATED APPLICATIONS

This application is a continuation application of International Application No. PCT/CN2023/091052 filed on Apr. 27, 2023, which claims priority to Chinese Patent Application No. 202210804792.2 filed with the China National Intellectual Property Administration on Jul. 8, 2022, the disclosures of each being incorporated by reference herein in their entireties.

### FIELD

The disclosure relates to the field of computer technology, and in particular, to an antigen prediction method and apparatus, a device, and a storage medium

### BACKGROUND

A human immune system is composed of innate immunity and adaptive immunity. An adaptive immune system is achieved through various immune cells, which exhibit specific responses to particular pathogens. Immune cell receptors are regions where immune cells recognize antigens, and successful recognition of antigens can activate the immune system to eliminate pathogens, playing a crucial role in maintaining human health.

Immune cell receptors possess antigen specificity, meaning that an immune cell receptor can only bind to a specific antigen. Investigating the antigen specificity of immune cell receptors is crucial for understanding an immune system and further advancing immune therapies, vaccine design, and development. Based on this, there is an urgent need for a method for predicting antigens that can specifically bind to immune cell receptors.

### SUMMARY

Some embodiments provide an antigen prediction method and apparatus, a device, and a storage medium.

Some embodiments provide an antigen prediction method including inputting genetic information, sequence information, and three-dimensional structure features of an immune cell receptor into an antigen prediction model; performing, by the antigen prediction model, feature extraction on the genetic information and the sequence information to obtain genetic features and sequence features of the immune cell receptor; integrating, by the antigen prediction model, the genetic features, the sequence features, and the three-dimensional structure features to obtain receptor features of the immune cell receptor; performing, by the antigen prediction model, full connection and normalization on the receptor features to output a probability of the immune cell receptor being associated with each candidate antigen of a plurality of candidate antigens; and determining, based on the probability of the immune cell receptor being associated with each candidate of the plurality of candidate antigens, an antigen binding to the immune cell receptor from the plurality of candidate antigens.

Some embodiments provide an antigen prediction apparatus including: at least one memory configured to store program code; and at least one processor configured to read the program code and operate as instructed by the program code, the program code comprising: input code configured to cause at least one of the at least one processor to input genetic information, sequence information, and three-dimensional structure features of an immune cell receptor into an antigen prediction model; feature extraction code configured to cause at least one of the at least one processor to perform, by the antigen prediction model, feature extraction on the genetic information and the sequence information to obtain genetic features and sequence features of the immune cell receptor; feature fusion code configured to cause at least one of the at least one processor to integrate, by the antigen prediction model, the genetic features, the sequence features, and the three-dimensional structure features to obtain receptor features of the immune cell receptor; and antigen prediction code configured to cause at least one of the at least one processor to perform, by the antigen prediction model, full connection and normalization on the receptor features to output a probability of the immune cell receptor being associated with each candidate antigen of a plurality of candidate antigens; and determining, based on the probability of the immune cell receptor being associated with each candidate of the plurality of candidate antigens, an antigen binding to the immune cell receptor from the plurality of candidate antigens.

Some embodiments provide a non-transitory computer-readable storage medium storing computer code which, when executed by at least one processor, causes the at least one processor to at least: input genetic information, sequence information, and three-dimensional structure features of an immune cell receptor into an antigen prediction model; perform, by the antigen prediction model, feature extraction on the genetic information and the sequence information to obtain genetic features and sequence features of the immune cell receptor; integrate, by the antigen prediction model, the genetic features, the sequence features, and the three-dimensional structure features to obtain receptor features of the immune cell receptor; perform, by the antigen prediction model, full connection and normalization on the receptor features to output a probability of the immune cell receptor being associated with each candidate antigen of a plurality of candidate antigens; and determine, based on the probability of the immune cell receptor being associated with each candidate of the plurality of candidate antigens, an antigen binding to the immune cell receptor from the plurality of candidate antigens.

### BRIEF DESCRIPTION OF THE DRAWINGS

To describe the technical solutions of some embodiments of this disclosure more clearly, the following briefly introduces the accompanying drawings for describing some embodiments. The accompanying drawings in the following description show only some embodiments of the disclosure, and a person of ordinary skill in the art may still derive other drawings from these accompanying drawings without creative efforts. In addition, one of ordinary skill would understand that aspects of some embodiments may be combined together or implemented alone.

FIG. 1 is a schematic diagram of an implementation environment of an antigen prediction method according to some embodiments.

FIG. 2 is a flowchart of an antigen prediction method according to some embodiments.

FIG. 3 is a flowchart of another antigen prediction method according to some embodiments.

FIG. 4 is a flowchart of determining three-dimensional structure features according to some embodiments.

FIG. 5 is a flowchart of another antigen prediction method according to some embodiments.

FIG. 6 is a schematic diagram of an experimental result according to some embodiments.

FIG. 7 is a flowchart of a method for training an antigen prediction model according to some embodiments.

FIG. 8 is a schematic structural diagram of an antigen prediction apparatus according to some embodiments.

FIG. 9 is a schematic structural diagram of an apparatus for training an antigen prediction model according to some embodiments.

FIG. 10 is a schematic structural diagram of a server according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure and the appended claims.

In the following descriptions, related “some embodiments” describe a subset of all possible embodiments. However, it may be understood that the “some embodiments” may be the same subset or different subsets of all the possible embodiments, and may be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

To more clearly describe the technical solutions of some embodiments, related terms are described herein.

Embedded Coding represents a mapping relationship mathematically, that is, data from a space X is mapped to a space Y through a function F, where the function F is an injective function, and a mapping result is structure preservation. The injective function implies a unique association between mapped data and data before mapping. The structure preservation means that the size relationship between the data before mapping is the same with the size relationship between the mapped data. For example, if data X₁ and X₂ exist before mapping, after mapping, Y₁ associated with X₁ and Y₂ associated with X₂ are obtained. If the data X₁ is greater than the data X₂ before mapping, the mapped data Y₁ is greater than the mapped data Y₂ correspondingly. For amino acids, the amino acids are mapped to another space, facilitating subsequent machine learning and processing.

Attention Weight represents the importance of specific data during training or prediction, and the importance signifies the degree to which input data influences output data. Data with higher importance has higher attention weights, while data with lower importance has lower attention weights. In different scenarios, the importance of data varies, and the process of training attention weights in a model is a process of determining the importance of data.

Immune Cells are commonly known as white blood cells, immune cells include innate lymphocytes, various phagocytes, lymphocytes capable of recognizing antigens and generating specific immune responses, etc.

T Cells are short for T-lymphocytes, and originate from pluripotent stem cells in the bone marrow (which originate from the yolk sac and liver in embryos). During embryonic and neonatal stages of the human body, some pluripotent stem cells or pre-T cells in the bone marrow migrate to the thymus, and differentiate and mature into T cells with immunological activity under the induction of thymic hormones.

TCR: T cell receptor (TCR) is a characteristic marker on surfaces of all T cells. The function of TCR is to recognize antigens.

B Cells are short for B lymphocytes, and originate from pluripotent stem cells in the bone marrow. The precursor cells of B lymphocytes exist in hematopoietic islands of the embryonic liver (around day 14 in mouse embryos or 8-9 weeks in vaginally delivered babies). Subsequently, the place for the production and differentiation of B lymphocytes is gradually replaced by the bone marrow. Mature B cells primarily reside within the superficial cortex lymphoid nodules of the lymph nodes and the lymphoid nodules within the red pulp and white pulp of the spleen. Upon antigen stimulation, B cells can differentiate into plasma cells, which synthesize and secrete antibodies (immunoglobulins), primarily executing humoral immunity in the body.

BCR: B-cell receptor (BCR) is a molecule located on the surface of B cells responsible for the specific recognition and binding of antigens, which is essentially surface membrane immunoglobulin. BCR possesses antigen binding specificity.

Antigen generally refers to any substance that can stimulate the body to produce a specific immune response (both humoral and cellular immunity).

Cloud technology refers to a hosting technology that integrates hardware, software, networks, and other resources within a wide area network or a local area network to realize computing, storage, processing, and sharing of data.

The technical solution provided in some embodiments may also be combined with the cloud technology. For example, a trained antigen prediction model is deployed on a cloud server. Medical cloud in cloud technology refers to the utilization of “cloud computing” in conjunction with the medical technology to create a healthcare service cloud platform based on new technologies such as cloud computing, mobile technology, multimedia, 4G communication, big data, and the Internet of Things, thereby sharing medical resources and expanding the medical ranges.

Information (including but not limited to user device information, user personal information, etc.), data (including but not limited to data for analysis, stored data, displayed data, etc.), and signals involved in some embodiments are all authorized by users or sufficiently authorized by each side, and related data collection, use and processing need to comply with relevant laws, regulations, and standards of related countries and regions. For example, genetic information involved in some embodiments is obtained with full authorization.

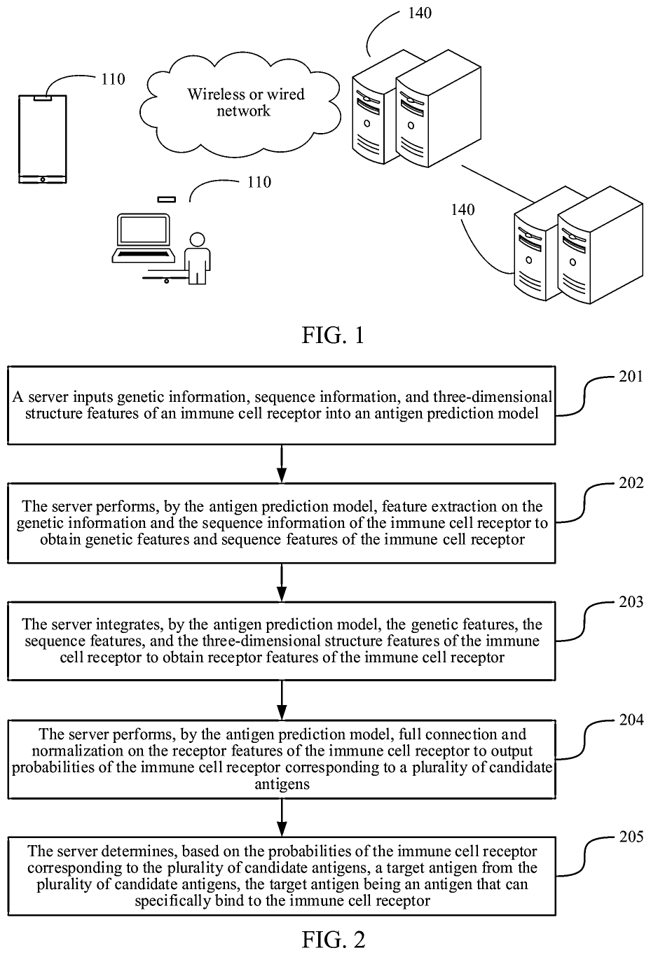

FIG. 1 is a schematic diagram of an implementation environment of an antigen prediction method according to some embodiments. Referring to FIG. 1, the implementation environment includes a terminal 110 and a server 140.

The terminal 110 is connected with the server 140 by using a wired network or wireless network. In some embodiments, the terminal 110 is a smart phone, a tablet, a laptop, a desktop computer, a smart watch, or the like, but is not limited them. An application that supports antigen prediction is installed and runs on the terminal 110.

The server 140 is an independent physical server, or a server cluster or distributed system formed by a plurality of physical servers, or a cloud server providing basic cloud computing services such as cloud services, cloud databases, cloud computing, cloud functions, cloud storage, network services, cloud communication, middleware services, domain name services, security services, content delivery network (CDN), big data, and an artificial intelligence platform.

Those skilled in the art understand that the number of the terminals and the servers is not limited to the above. For example, there may be only one terminal as mentioned above, or there may be dozens or hundreds of terminals as mentioned above, or even more. In this case, the above implementation environment also includes other terminals. The number of the terminals and the device type are not limited thereto.

In the following description process, the terminal refers to the terminal 110 in the above implementation environment, and the server refers to the server 140 in the above implementation environment.

The antigen prediction method provided by some embodiments can be applied to the fields such as scientific research and vaccine design, namely, scenarios where the antigen specificity of an immune cell receptor is determined. The antigen specificity refers to the ability of specifically binding to the immune cell receptor by a target antigen. Through the technical solution provided by some embodiments, technical personnel upload genetic information, sequence information, and three-dimensional structure features of the immune cell receptor to the server through the terminal. The server processes the genetic information, the sequence information, and the three-dimensional structure features of the immune cell receptor using the trained antigen prediction model, thereby obtaining receptor features of the immune cell receptor. The genetic information of the immune cell receptor includes VDJ information of the immune cell receptor, the sequence information represents an amino acid sequence of the immune cell receptor, and the three-dimensional structure features represent a three-dimensional structure of the immune cell receptor. Based on the receptor features of the immune cell receptor, the server performs antigen prediction using the antigen prediction model, and outputs the target antigen corresponding to the immune cell receptor. The target antigen refers to an antigen that can specifically bind to the immune cell receptor. Technical personnel can further conduct scientific research or vaccine design based on the target antigen. By adopting the technical solution provided by some embodiments, the number of experiments conducted by technical personnel based on the immune cell receptor can be reduced, thereby improving the efficiency of scientific research and vaccine design

The technical solutions provided by some embodiments are implemented by a computer device, such as a terminal or a server, or a combination of a terminal and a server. Both the terminal and the server are used as examples for the computer device. In the following description process, the server is used as an example of an executing entity, and referring to FIG. 2, the method includes the following operations.

201. A server inputs genetic information, sequence information, and three-dimensional structure features of an immune cell receptor into an antigen prediction model.

The immune cell receptor is a T cell receptor or a B-cell receptor. In some embodiments, the genetic information of the immune cell receptor includes VDJ information of the immune cell receptor, where V represents an encoding variable region, D represents an encoding hypervariable region, and J represents an encoding joining region. The sequence information of the immune cell receptor represents an amino acid sequence of the immune cell receptor. The three-dimensional structure features of the immune cell receptor are determined based on a three-dimensional structure of the immune cell receptor, where the three-dimensional structure is used for representing the positions of a plurality of amino acids in the immune cell receptor. The three-dimensional structure features can overall reflect the three-dimensional structure of the immune cell receptor. The antigen prediction model is a model trained based on the genetic information, the sequence information, and the three-dimensional structure features of the sample immune cell receptor, and has the function of predicting the corresponding antigen for the immune cell receptor. For example, the antigen prediction model can at least predict the probability of the inputted immune cell receptor being associated with a candidate antigen. The probability represents the likelihood of the immune cell receptor being associated with the candidate antigen, or the probability represents the likelihood of expected specific binding between the candidate antigen and the immune cell receptor.

202. The server performs feature extraction on the genetic information and the sequence information of the immune cell receptor through the antigen prediction model to obtain genetic features and sequence features of the immune cell receptor.

The process of performing the feature extraction on the genetic information and the sequence information of the immune cell receptor is a process of performing abstract expression on the genetic information and the sequence information of the immune cell receptor. The obtained genetic features and sequence features can not only represent the genetic information and sequence information of the immune cell receptor, but also facilitate further processing by the server.

The genetic features are features extracted based on the genetic information and represent the features of the VDJ information of the immune cell receptor. Likewise, the sequence features are features extracted based on the sequence information and represent the features of the amino acid sequence of the immune cell receptor

In some embodiments, the antigen prediction model is used for performing the feature extraction on the genetic information of the immune cell receptor to obtain the genetic features of the immune cell receptor; and the antigen prediction model is used for performing the feature extraction on the sequence information of the immune cell receptor to obtain the sequence features of the immune cell receptor.

203. The server integrates, by the antigen prediction model, the genetic features, the sequence features, and the three-dimensional structure features of the immune cell receptor, thereby obtaining receptor features of the immune cell receptor.

The receptor features of the immune cell receptor are obtained by integrating the genetic features, the sequence features, and the three-dimensional structure features, allowing the representation of the immune cell receptor from three aspects including the gene, sequence and structure. Therefore, the receptor features possess strong expression capabilities. In other words, the receptor features are used for representing integrated features (or global features) of the immune cell receptor from three aspects including the gene, sequence and structure.

204. The server performs, by the antigen prediction model, full connection and normalization on the receptor features of the immune cell receptor to output probabilities of the immune cell receptor corresponding to a plurality of candidate antigens.

The process of performing the full connection and normalization based on the receptor features of the immune cell receptor is a process of performing antigen prediction based on the receptor features of the immune cell receptor.

In operation 204, the server is pre-configured with a plurality of candidate antigens. The candidate antigens may be antigens filtered, by technical personnel or algorithms, from natural antigens in the natural environment or antigens synthesized through chemical means. The antigen prediction model performs the full connection and normalization on the receptor features of the immune cell receptor and outputs the probability of the immune cell receptor being associated with each candidate antigen. The probability represents the likelihood of the immune cell receptor being associated with the candidate antigen, or, the probability represents the likelihood of expected specific binding between the candidate antigen and the immune cell receptor.

205. The server determines, based on the probabilities of the immune cell receptor corresponding to the plurality of candidate antigens, a target antigen from the plurality of candidate antigens, and the target antigen is an antigen that can specifically bind to the immune cell receptor.

In operation 205, the server determines, based on the probability of the immune cell receptor being associated with each candidate antigen, the antigen that can specifically bind to the immune cell receptor from the plurality of candidate antigens. Therefore, the antigen that can specifically bind to the immune cell receptor can be screened from the plurality of candidate antigens, facilitating guiding subsequent scientific research or vaccine design.

In some embodiments, a human immune system is composed of innate immunity and adaptive immunity. The adaptive immunity is an immune response that can recognize, upon exposure to an antigen (specific pathogen), the antigen, and is initiated for the antigen. The inputted immune cell receptor in operation 201 and the predicted antigen in operation 205 form a pair of “receptor-antigen” expected by a machine to be capable of generating specific binding. However, this pair of “receptor-antigen” pair (i.e., the immune cell receptor in operation 201 and the predicted antigen in operation 205) needs to be further subjected to biological experiments to validate whether the immune cell receptor in operation 201 and the predicted antigen in operation 205 can exhibit specific binding, thereby assisting in scientific research or vaccine design based on experimental results.

For example, T cells and B cells are important components of an adaptive immune system. Antigen recognition is one of key factors in the immune response mediated by the T cells and the B cells. T cell immunity is mainly mediated by the interaction between a T cell receptor (TCR, a kind of protein dimer) and the antigen, and B cell immunity is mainly mediated by the interaction between a B-cell receptor (BCR) and the antigen.

Based on this, by inspecting the scenario of T cell antigen prediction, immune cells refer to the T cells, the immune cell receptor involved in operation 201 refers to the T cell receptor, and the antigen predicted in operation 205 refers to the antigen that is expected by the machine to be capable of specifically binding to the T cell receptor (referred to as a T cell antigen later). Therefore, technical personnel conduct the biological experiment on the T cell receptor and the T cell antigen. The aforementioned biological experiment includes: observing a success rate (activation rate) of T cells activated with immune response upon stimulation by the T cell antigen. The aforementioned success rate/activation rate may be, for example, a ratio/percentage obtained by dividing the total number of T cells activated with immune response by the total number of the T cells (i.e., the total number of T cell samples used in the biological experiment), where the T cells activated with immune response are also known as activated T cells. In some embodiments, the aforementioned success rate/activation rate may include a recognition success rate of the T cell receptor recognizing the T cell antigen, as well as an initiation success rate of the immune response against the T cell antigen by the T cells upon stimulation by the T cell antigen. The recognition success rate is obtained by dividing the number of T cells where the T cell receptor successfully recognizes the T cell antigen by the aforementioned total number of the T cells, and the initiation success rate is obtained by dividing the number of activated T cells by the aforementioned total number of the T cells. In some embodiments, the above recognition success rate may be derived/reversely derived from the above initiation success rate. For example, assuming that all T cells that successfully recognize the T cell antigen will be activated with immune response, the above recognition success rate equals the initiation success rate. In some embodiments, the above recognition success rate and the above initiation success rate can be measured respectively. The method has a transformative impact on the fields such as disease treatment, vaccine design, and scientific research. Due to the expression of specific molecules on the surfaces of the activated T cells, by measuring the types and number of molecular expressions on the surfaces of the T cells within a set time period after applying T cell antigen stimulation, whether the T cells are activated with immune response or whether the T cells are activated can be judged, and in other words, whether the T cells generate the immune response is judged. Specific molecules expressed on the surfaces of the activated T cells include but not limited to: early activation marker CD69 molecules, mid-stage activation marker CD25 molecules, late-stage activation marker CD71, CD38, and HLA-DR molecules, etc.

Similarly, by inspecting the scenario of B cell antigen prediction, immune cells refer to the B cells, the immune cell receptor involved in operation 201 refers to the B-cell receptor, and the antigen predicted in operation 205 refers to the antigen that can specifically bind to the B-cell receptor (referred to as a B cell antigen later) through machine prediction. Therefore, technical personnel conduct the biological experiment on the B-cell receptor and the B cell antigen. The aforementioned biological experiment includes: observing a success rate (activation rate) of B cells activated with immune response upon stimulation by the B cell antigen. The aforementioned success rate/activation rate may be, for example, a ratio/percentage obtained by dividing the total number of B cells activated with immune response by the total number of the B cells (i.e., the total number of B cell samples used in the biological experiment), where the B cells activated with immune response are also known as activated B cells. In some embodiments, the aforementioned success rate/activation rate may include a recognition success rate of the B-cell receptor recognizing the B cell antigen, as well as an initiation success rate of the immune response against the B cell antigen by the B cells upon stimulation by the T cell antigen. The recognition success rate is obtained by dividing the number of B cells where the B-cell receptor successfully recognizes the B cell antigen by the aforementioned total number of the B cells, and the initiation success rate is obtained by dividing the number of activated B cells by the aforementioned total number of the B cells. In some embodiments, the above recognition success rate may be derived/reversely derived from the above initiation success rate. For example, assuming that all B cells that successfully recognize the B cell antigen will be activated with immune response, the above recognition success rate equals the initiation success rate. In some embodiments, the above recognition success rate and the above initiation success rate can be measured respectively. The method has a transformative impact on the fields such as disease treatment, vaccine design, and scientific research. Due to the expression of specific molecules on the surfaces of the activated B cells, by measuring the types and number of molecular expressions on the surfaces of the B cells within a set time period after applying B cell antigen stimulation, whether the B cells are activated with immune response or whether the B cells are activated can be judged, and in other words, whether the B cells generate the immune response is judged. Specific molecules expressed on the surfaces of the activated B cells include but not limited to: early activation marker CD69 molecules, mid-stage activation marker CD25 molecules, late-stage activation markers CD71 molecules, etc. CD38 and HLA-DR molecules cannot be used as detection indicators for B cell activation, and are only used as detection indicators for T cell activation.

Through the technical solution provided by some embodiments, the antigen prediction model performs the feature extraction on the genetic information and sequence of the immune cell receptor to obtain the genetic features and the sequence features of the immune cell receptor. In the process of obtaining the receptor features of the immune cell receptor, the genetic features, the sequence features, and the three-dimensional structure features are integrated. The introduction of the three-dimensional structure features enriches the content of the receptor features and enhances the expression capability of the receptor features. Therefore, when antigen prediction is performed based on the receptor features, the accuracy of the obtained target antigen is high.

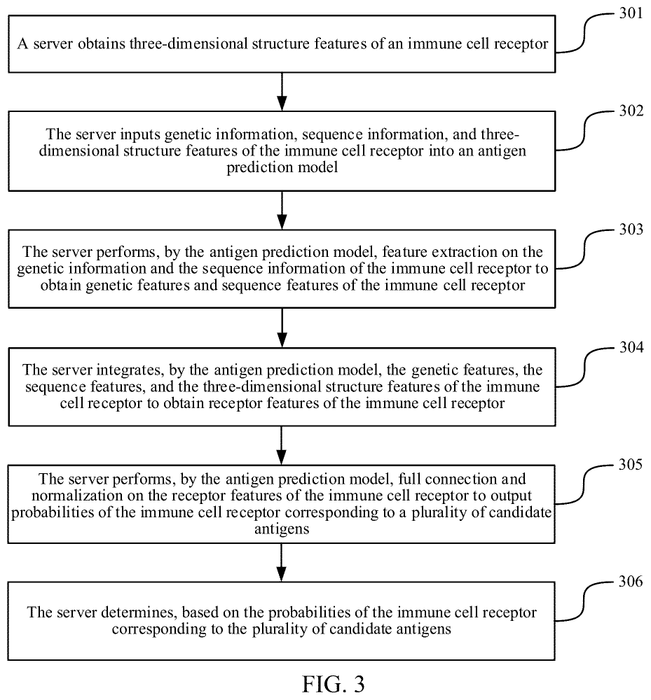

Operations 201 to 205 mentioned above provide a brief description of the antigen prediction method provided by some embodiments. The antigen prediction method provided by some embodiments is further described in conjunction with some examples as below. Referring to FIG. 3, the method is executed primarily by a computer device, and taking the computer device being a server as an example, the method includes the following operations.

301. The server obtains three-dimensional structure features of an immune cell receptor.

The immune cell receptor is a T cell receptor or a B-cell receptor. The immune cell receptor is used for recognizing an antigen and specifically binding to the antigen, thereby activating an immune system. The immune cell receptor is a kind of protein including multiple amino acids, and the three-dimensional structure features of the immune cell receptor are used for representing the spatial positions of the multiple amino acids of the immune cell receptor.

In some embodiments, the server obtains a target amino acid sequence of the immune cell receptor, and the target amino acid sequence includes a CDR3 region of the immune cell receptor. The server performs multiple sequence alignment on the target amino acid sequence of the immune cell receptor to obtain at least one reference amino acid sequence. The similarity between the reference amino acid sequence and the target amino acid sequence satisfies a similarity condition. The server obtains a homologous template of the target amino acid sequence. The homologous template includes structural information of homologous sequences of the target amino acid sequence. The server performs, based on the target amino acid sequence, the at least one reference amino acid sequence, and the homologous template, multiple rounds of iterations to obtain the three-dimensional structure features of the immune cell receptor.

In other words, the server obtains an amino acid sequence containing the CDR3 region of the immune cell receptor; the multiple sequence alignment is performed on the amino acid sequence to obtain at least one reference amino acid sequence, and the similarity between the reference amino acid sequence and the amino acid sequence satisfies a similarity condition; a homologous template of the amino acid sequence is obtained, and includes structural information of homologous sequences of the amino acid sequence; and multiple rounds of iterations are performed based on the amino acid sequence, the at least one reference amino acid sequence, and the homologous template to obtain three-dimensional structure features of the immune cell receptor.

A complementary determining region (CDR) exists on the immune cell receptor, and includes three sub-regions: CDR1, CDR2, and CDR3, where CDR3 exhibits the highest variability and plays a crucial role in antigen recognition.

In some embodiments, the server can determine the three-dimensional structure features of the immune cell receptor based on the target amino acid sequence of the immune cell receptor without the need for other devices such as cryo-electron microscopy for observation, thereby improving the efficiency of obtaining the three-dimensional structure features and reducing the cost of obtaining the three-dimensional structure features.

For example, the server obtains sequencing data of the immune cell receptor. The sequencing data includes a plurality of amino acids of the immune cell receptor and an arrangement order of the plurality of amino acids. The sequencing data is tested through a gene sequencing device by technical personnel, which is not limited herein. The server performs data preprocessing on the sequencing data of the immune cell receptor to obtain reference sequencing data of the immune cell receptor. The preprocessing on the sequencing data includes removing wrong data in the sequencing data and converting the sequencing data into a format easier for the server to process, and a preprocessing rule is set by technical personnel according to actual situations, which is not limited herein. The server performs quality control on the reference sequencing data to obtain target sequencing data of the immune cell receptor, where the quality control on the reference sequencing data includes filtering out dead cells, background estimation, paired chains, dextramer signal correction, Log-rank test, receptor gene clustering, etc. The server truncates a target length of amino acid sequence containing the CDR3 region from the target sequencing data. The target length of amino acid sequence containing the CDR3 region is the target amino acid sequence, where the target length is set by technical personnel according to actual situations, for example, the target length is set to be greater than 50 amino acids, which is not limited herein. In a case that the similarity condition is the similarity between amino acid sequences is greater than or equal to a similarity threshold, the server searches a gene database based on the target amino acid sequence to obtain at least one reference amino acid sequence. The reference amino acid sequence is an amino acid sequence having a similarity with the target amino acid sequence greater than or equal to the similarity threshold. The similarity between the amino acid sequences is determined by comparing the types and arrangement orders of amino acids in the amino acid sequences. Multiple sequence alignment, also known as multi-sequence alignment, is used for extracting and inputting sequences similar to the amino acid sequences from a large database and performing alignment simultaneously. The similarity threshold is a parameter pre-configured by technical personnel or a default value. Since amino acid sequences with similar sequences usually have similar folding patterns, by performing multiple sequence alignment, similar sequence structure information can be incorporated into the features. The server obtains a homologous template corresponding to the target amino acid sequence by searching a structure database based on the target amino acid sequence. The homologous template includes structural information of homologous sequences of the target amino acid sequence. The server performs, based on an attention mechanism, multiple rounds of iterative encoding on the target amino acid sequence, the at least one reference amino acid sequence, and the homologous template to obtain distance distribution between each pair of amino acids in the target amino acid sequence and the angles of chemical bonds connecting the amino acids. By using the attention mechanism, the server encodes the distance distribution between each pair of amino acids in the target amino acid sequence and the angles of the chemical bonds connecting the amino acids to output three-dimensional structure information of the immune cell receptor. The three-dimensional structure information of the immune cell receptor includes three-dimensional positions of the plurality of amino acids in the immune cell receptor. The server performs feature extraction on the three-dimensional structure information of the immune cell receptor, such as using a graph network to process the three-dimensional structure information of the immune cell receptor, to obtain the three-dimensional structure features of the immune cell receptor.

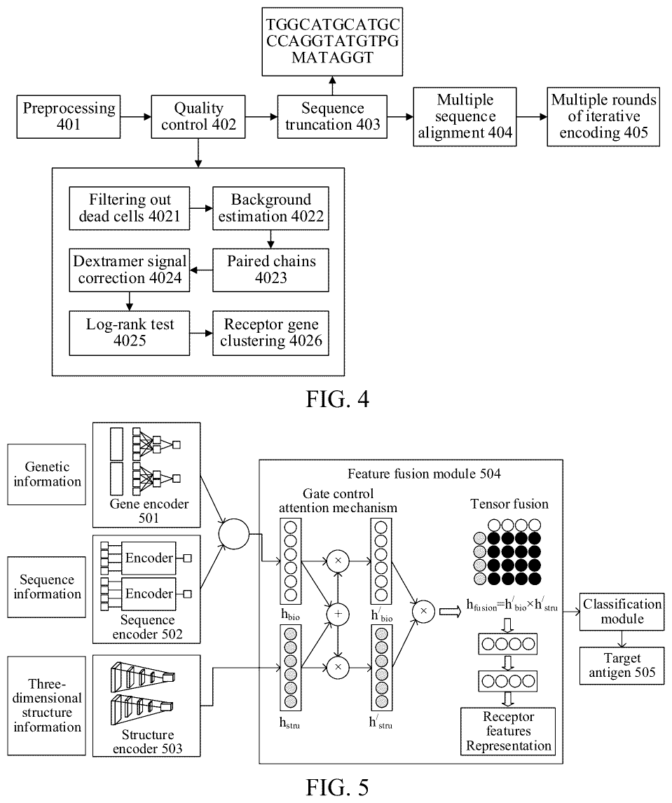

To provide a clearer description of the above implementation, the above implementation is described in conjunction with FIG. 4 as below.

Referring to FIG. 4, the server performs preprocessing 401 on sequencing data of the immune cell receptor to obtain reference sequencing data of the immune cell receptor The server performs quality control 402 on the reference sequencing data to obtain target sequencing data of the immune cell receptor, where the quality control 402 includes filtering out dead cells 4021, background estimation 4022, paired chains 4023, dextramer signal correction 4024, Log-rank test 4025, and receptor gene clustering 4026. The server performs sequence truncation 403 on the target sequencing data to obtain a target amino acid sequence. The server performs multiple sequence alignment 404 based on the target amino acid sequence to obtain at least one reference amino acid sequence. The server obtains a homologous template corresponding to the target amino acid sequence by searching a structure database based on the target amino acid sequence. The server performs, based on the attention mechanism, multiple rounds of iterative encoding 405 on the target amino acid sequence, the at least one reference amino acid sequence, and the homologous template to obtain three-dimensional structure information of the immune cell receptor, and performs feature extraction on the three-dimensional structure information to obtain three-dimensional structure features of the immune cell receptor.

The above implementation is a method that the server determines the three-dimensional structure features of the immune cell receptor based on the target amino acid sequence of the immune cell receptor. In some embodiments, the server utilizes trained structure prediction models to obtain the three-dimensional structure features based on the amino acid sequence. The structure prediction models include models such as RoseTTAFold, AlphaFold, and AlphaFold2. Certainly, with the advancement of science and technology, other structure prediction models may also be adopted, which are not limited herein. The structure prediction model is configured to extract the three-dimensional structure features of the immune cell receptor based on the inputted amino acid sequence of the immune cell receptor.

The method that the server obtains the three-dimensional structure features of the immune cell receptor based on the three-dimensional structure information of the immune cell receptor is described as below. The three-dimensional structure information includes three-dimensional positions (e.g., three-dimensional coordinates) of a plurality of amino acids in the immune cell receptor.

In some embodiments, the server obtains three-dimensional structure information of the immune cell receptor, and the three-dimensional structure information includes three-dimensional coordinates of a plurality of amino acids in the immune cell receptor. The server performs graph convolution on the three-dimensional structure information of the immune cell receptor to obtain three-dimensional structure features of the immune cell receptor.

The three-dimensional structure information refers to a three-dimensional structure file of the immune cell receptor. In some embodiments, the three-dimensional structure information is obtained from images captured using cryo-electron microscopy, or obtained by a structure prediction model based on the amino acid sequence of the immune cell receptor, which is not limited herein. Graph convolution is short for graph convolutional network (GCN), and is used for extracting features of a graph. In some embodiments, nodes in the graph represent amino acids in the immune cell receptor, edges in the graph are used for representing the relative positional relationships between the amino acids, and the edge herein refers to connecting edges between any two nodes in the graph.

In some embodiments, the server directly performs graph convolution on the three-dimensional structure information of the immune cell receptor to obtain the three-dimensional structure features of the immune cell receptor without pre-determining the three-dimensional structure information of the immune cell receptor, such that the efficiency of determining the three-dimensional structure features is high.

For example, the server obtains three-dimensional structure information of the immune cell receptor. The server generates a three-dimensional structure graph of the immune cell receptor based on the three-dimensional structure information. Each node in the three-dimensional structure graph corresponds to an amino acid in the immune cell receptor, and edges in the three-dimensional structure graph are used for representing connection relationships between the amino acids. Node features of the nodes in the three-dimensional structure graph include the types and three-dimensional coordinates of the corresponding amino acids. The server performs graph convolution on the three-dimensional structure graph to obtain three-dimensional structure features of the immune cell receptor. In other words, each node in the three-dimensional structure graph indicates an amino acid in the immune cell receptor, each edge in the three-dimensional structure graph is used for connecting two nodes, and the edge represents the relative positional relationship between the two amino acids respectively indicated by the two nodes, or represents the connection relationship between the two amino acids respectively indicated by the two nodes. Additionally, modeling is performed on the node features of each node, and the type and the three-dimensional coordinates of the amino acid indicated by each node serve as the node features of the node.

In some embodiments, the server obtains three-dimensional structure information of the immune cell receptor, and the three-dimensional structure information includes three-dimensional coordinates of a plurality of amino acids in the immune cell receptor. The server encodes, based on the attention mechanism, the three-dimensional structure information of the immune cell receptor to obtain three-dimensional structure features of the immune cell receptor.

In some embodiments, the server can obtain the three-dimensional structure features of the immune cell receptor by directly encoding the three-dimensional structure information of the immune cell receptor based on the attention mechanism without pre-determining the three-dimensional structure information of the immune cell receptor, such that the efficiency of determining the three-dimensional structure features is high.

For example, in some embodiments, the server obtains three-dimensional structure information of the immune cell receptor. The server performs embedded encoding on a plurality of amino acids in the three-dimensional structure information to obtain embedding features of the plurality of amino acid. The process of embedded encoding of the plurality of amino acids is a process of discrete representation of the plurality of amino acids, facilitating subsequent processing by the server. By using the attention mechanism, the server encodes the embedding features of the plurality of amino acids based on the three-dimensional structure information, thereby obtaining attention weights of the plurality of amino acids. The server integrates, based on the attention weights of the plurality of amino acids, the embedding features of the plurality of amino acids to obtain three-dimensional structure features of the immune cell receptor. In some embodiments, the server can adopt an encoder of a Transformer model to encode the three-dimensional structure information of the immune cell receptor so as to obtain the three-dimensional structure features of the immune cell receptor. In other words, each amino acid in the three-dimensional structure information is subjected to embedded encoding, thereby obtaining the amino acid embedding features of each amino acid. Then, by using the attention mechanism, the amino acid embedding features of each amino acid are encoded based on the three-dimensional structure information, thereby obtaining the attention weight of each amino acid. Subsequently, based on the attention weights of the amino acids, the amino acid embedding features of the amino acids are integrated to obtain the three-dimensional structure features of the immune cell receptor. For example, the mentioned integrating method refers to a weighted summation, where the attention weight of each amino acid serves as a weighted coefficient for the amino acid embedding features of each amino acid.

The above embodiments are provided as examples where the server utilizes the graph convolution and the attention mechanism for encoding the three-dimensional structure information of the immune cell receptor and obtaining the three-dimensional structure features. In some embodiments, the server may also adopt other models to encode the three-dimensional structure information of the immune cell receptor, which is not limited herein.

In some embodiments, operation 301 may be an optional operation.

302. The server inputs genetic information, sequence information, and the three-dimensional structure features of the immune cell receptor into an antigen prediction model.

The genetic information of the immune cell receptor includes VDJ information of the immune cell receptor, where V represents an encoding variable region, D represents an encoding hypervariable region, and J represents an encoding joining region. The sequence information of the immune cell receptor represents the amino acid sequence of the immune cell receptor. For example, AEGAL represents an amino acid sequence, where A represents alanine, E represents glutamic acid, G represents glycine, and L represents leucine. The immune cell receptor is a kind of protein, and the amino acid sequence is also referred to as a one-dimensional structure of the protein. The antigen prediction model is a model trained based on the genetic information, the sequence information, and the three-dimensional structure features of a sample immune cell receptor, and has the function of predicting a corresponding antigen for the immune cell receptor.

In some embodiments, the antigen prediction model includes three information encoding channels. The first information encoding channel is a genetic information encoding channel, and the genetic information encoding channel includes a gene encoder configured to encode the genetic information; the second information encoding channel is a sequence information encoding channel, and the sequence information encoding channel includes a sequence encoder configured to encode the sequence information; and the third information encoding channel is a structure feature encoding channel, and the structure feature encoding channel includes a structure encoder configured to encode the structure features. The server inputs the genetic information of the immune cell receptor into the genetic information encoding channel of the antigen prediction model, and subsequently encodes the genetic information through the gene encoder in the genetic information encoding channel. The server inputs the sequence information of the immune cell receptor into the sequence information encoding channel of the antigen prediction model, and subsequently encodes the sequence information through the sequence encoder in the sequence information encoding channel. The server inputs the three-dimensional structure features of the immune cell receptor into the structure feature encoding channel, and subsequently encodes the three-dimensional structure features through the structure encoder in the structure feature encoding channel.

In some embodiments, before inputting the sequence information of the immune cell receptor into the antigen prediction model, the server can further preprocess the sequence information of the immune cell receptor to ensure that the length of the sequence information inputted into the antigen prediction model is uniform. In a case that the length of the sequence information of the immune cell receptor is greater than a length threshold, the server truncates the part of the sequence information of the immune cell receptor where the length is greater than or equal to the length threshold, thereby obtaining sequence information with the length being the length threshold, and subsequently, the truncated sequence information is inputted into the antigen prediction model. In a case that the length of the sequence information of the immune cell receptor is less than the length threshold, the server pads the sequence information of the immune cell receptor with a target symbol to obtain sequence information with the length being the length threshold, and subsequently inputs the truncated sequence information into the antigen prediction model, where the target symbol is set by technical personnel according to actual situations, such as 0. The length threshold is set by technical personnel according to actual situations.

Operation 301 and operation 302 are provided as an example to illustrate that the server obtains the three-dimensional structure features of the immune cell receptor in advance. In some embodiments, the server obtains the three-dimensional structure information of the immune cell receptor in advance, inputs the three-dimensional structure information into the structure feature encoding channel of the antigen prediction model, and subsequently, obtains the three-dimensional structure features of the immune cell receptor through the structure encoder of the structure feature encoding channel, which is not limited herein.

Additionally, operation 301 and operation 302 are provided as an example to illustrate that the server obtains the three-dimensional structure features of the immune cell receptor and inputs the genetic information, the sequence information, and the three-dimensional structure features of the immune cell receptor into the antigen prediction model. In some embodiments, in a case that the server does not obtain the three-dimensional structure features of the immune cell receptor, the server can only input the genetic information and the sequence information of the immune cell receptor to the antigen prediction model.

303. The server performs feature extraction on the genetic information and the sequence information of the immune cell receptor through the antigen prediction model to obtain genetic features and sequence features of the immune cell receptor.

The process of performing the feature extraction on the genetic information and the sequence information of the immune cell receptor is a process of performing abstract expression on the genetic information and the sequence information of the immune cell receptor. The obtained genetic features and sequence features can not only represent the genetic information and sequence information of the immune cell receptor, but also facilitate further processing by the server.

In some embodiments, the antigen prediction model includes a gene encoder and a sequence encoder. In a case that the genetic information includes VDJ information of the immune cell receptor, the server encodes, by the gene encoder of the antigen prediction model, the VDJ information of the immune cell receptor to obtain genetic features of the immune cell receptor, where V represents an encoding variable region, D represents an encoding hypervariable region, and J represents an encoding joining region. In a case that the sequence information includes an amino acid sequence of the immune cell receptor, the server encodes, by the sequence encoder of the antigen prediction model, the amino acid sequence of the immune cell receptor to obtain sequence features of the immune cell receptor.

In some embodiments, the server can encode the genetic information and the sequence information of the immune cell receptor respectively through the gene encoder and the sequence encoder of the antigen prediction model, that is, feature extraction is performed on the genetic information and the sequence information, and obtained genetic features and sequence features can represent the immune cell receptor from different dimensions.

To provide a clearer description of the above implementation, the above implementation is described in two parts.

First part: The server encodes, by the gene encoder of the antigen prediction model, the VDJ information of the immune cell receptor to obtain the genetic features of the immune cell receptor.

In some embodiments, in a case that the immune cell receptor is a B-cell receptor, the server encodes, by the gene encoder of the antigen prediction model, VJ information of light chains and VDJ information of heavy chains of the immune cell receptor to obtain genetic features of the immune cell receptor.

The B-cell receptor includes two identical heavy chains (H chains) and two identical light chains (L chains), and the two heavy chains and the two light chains are connected by interchain disulfide bonds to form a tetrameric structure. The heavy chain has a molecular weight of approximately 50-75 kDa, and is composed of 450-550 amino acid residues. The light chain has a molecular weight of approximately 25 kDa, and is composed of 214 amino acid residues.

To provide a clearer description of the above implementation, the above implementation is described by three examples.

Example 1: The server performs, by the gene encoder of the antigen prediction model, full connection on the VJ information of the light chains and the VDJ information of the heavy chains of the immune cell receptor to obtain the genetic features of the immune cell receptor, and the genetic features of the immune cell receptor include light chain genetic features and heavy chain genetic features of the immune cell receptor.

In some embodiments, the antigen prediction model includes two gene encoders. The server concatenates, by the first gene encoder of the antigen prediction model, the VJ information of the light chains of the B-cell receptor to obtain light chain genetic information of the B-cell receptor. The server concatenates, by the second gene encoder of the antigen prediction model, the VDJ information of the heavy chains of the B-cell receptor to obtain heavy chain genetic information of the B-cell receptor. The server performs, by the first gene encoder of the antigen prediction model, two-time full connection on the light chain genetic information of the B-cell receptor to obtain light chain genetic features of the B-cell receptor. The server performs, by the second gene encoder of the antigen prediction model, two-time full connection on the heavy chain genetic information of the B-cell receptor to obtain heavy chain genetic features of the B-cell receptor. The light chain genetic features and the heavy chain genetic features of the B-cell receptor constitute the genetic features of the B-cell receptor.

Example 2: The server performs, by the gene encoder of the antigen prediction model, convolution on the VJ information of the light chains and the VDJ information of the heavy chains of the immune cell receptor to obtain the genetic features of the immune cell receptor, and the genetic features of the immune cell receptor include light chain genetic features and heavy chain genetic features of the immune cell receptor.

In some embodiments, the antigen prediction model includes two gene encoders. The server concatenates, by the first gene encoder of the antigen prediction model, the VJ information of the light chains of the B-cell receptor to obtain light chain genetic information of the B-cell receptor. The server concatenates, by the second gene encoder of the antigen prediction model, the VDJ information of the heavy chains of the B-cell receptor to obtain heavy chain genetic information of the B-cell receptor. The server performs, by the first gene encoder of the antigen prediction model, two-time convolution on the light chain genetic information of the B-cell receptor to obtain light chain genetic features of the B-cell receptor. The server performs, by the second gene encoder of the antigen prediction model, two-time convolution on the heavy chain genetic information of the B-cell receptor to obtain heavy chain genetic features of the B-cell receptor. The light chain genetic features and the heavy chain genetic features of the B-cell receptor constitute the genetic features of the B-cell receptor.

Example 3: The server encodes, by the gene encoder of the antigen prediction model, the VJ information of the light chains and the VDJ information of the heavy chains of the immune cell receptor based on the attention mechanism to obtain the genetic features of the immune cell receptor, and the genetic features of the immune cell receptor include light chain genetic features and heavy chain genetic features of the immune cell receptor.

In some embodiments, the antigen prediction model includes two gene encoders. The server concatenates, by the first gene encoder of the antigen prediction model, the VJ information of the light chains of the B-cell receptor to obtain light chain genetic information of the B-cell receptor. The server concatenates, by the second gene encoder of the antigen prediction model, the VDJ information of the heavy chains of the B-cell receptor to obtain heavy chain genetic information of the B-cell receptor. The server encodes, by the first gene encoder of the antigen prediction model, the light chain genetic information of the B-cell receptor based on the attention mechanism to obtain light chain genetic features of the B-cell receptor. The server encodes, by the second gene encoder of the antigen prediction model, the heavy chain genetic information of the B-cell receptor based on the attention mechanism to obtain heavy chain genetic features of the B-cell receptor. The light chain genetic features and the heavy chain genetic features of the B-cell receptor constitute the genetic features of the B-cell receptor.

The above content is illustrated by using the immune cell receptor being the B-cell receptor as an example. The following is an example of using the immune cell receptor being a T cell receptor for illustration.

In some embodiments, in a case that the immune cell receptor is the T cell receptor, the server encodes, by the gene encoder of the antigen prediction model, VJ information of an α chain and VDJ information of a β chain of the immune cell receptor to obtain genetic features of the immune cell receptor.

Some T cell receptors include an α chain and a β chain, and this type of T cell receptor is also referred to as αβ-TCR. Some other T cell receptors include a γ chain and a δ chain, and this type of T cell receptor is also referred to as γδ-TCR. Since the number of αβ-TCRs in the human body is much greater than the number of γδ-TCRs, in the following description process, the T cell receptor being αβ-TCR is used as an example for illustration. The structure of γδ-TCR and the structure of αβ-TCR are similar and both are a dual-chain structure. The processing method belongs to the same disclosure concept, and for the implementation process, reference is made to the following description.

To provide a clearer description of the above implementation, the above implementation is described by three examples.

Example 1: The server performs, by the gene encoder of the antigen prediction model, full connection on the VJ information of the α chain and the VDJ information of the β chain of the immune cell receptor to obtain genetic features of the immune cell receptor, and the genetic features of the immune cell receptor include α chain genetic features and β chain genetic features of the immune cell receptor.

In some embodiments, the antigen prediction model includes two gene encoders. The server concatenates, by the first gene encoder of the antigen prediction model, the VJ information of the α chain of the T cell receptor to obtain α chain genetic information of the T cell receptor. The server concatenates, by the second gene encoder of the antigen prediction model, the VDJ information of the β chain of the T cell receptor to obtain β chain genetic information of the T cell receptor. The server performs, by the first gene encoder of the antigen prediction model, two-time full connection on the α chain genetic information of the T cell receptor to obtain α chain genetic features of the T cell receptor. The server performs, by the second gene encoder of the antigen prediction model, two-time full connection on the β chain genetic information of the T cell receptor to obtain β chain genetic features of the T cell receptor. The α chain genetic features and the β chain genetic features of the T cell receptor constitute the genetic features of the T cell receptor.

Example 2: The server performs, by the gene encoder of the antigen prediction model, convolution on the VJ information of the α chain and the VDJ information of the β chain of the immune cell receptor to obtain genetic features of the immune cell receptor, and the genetic features of the immune cell receptor include α chain genetic features and β chain genetic features of the immune cell receptor.

In some embodiments, the antigen prediction model includes two gene encoders. The server concatenates, by the first gene encoder of the antigen prediction model, the VJ information of the α chain of the T cell receptor to obtain α chain genetic information of the T cell receptor. The server concatenates, by the second gene encoder of the antigen prediction model, the VDJ information of the β chain of the T cell receptor to obtain β chain genetic information of the T cell receptor. The server performs, by the first gene encoder of the antigen prediction model, two-time convolution on the α chain genetic information of the T cell receptor to obtain α chain genetic features of the T cell receptor. The server performs, by the second gene encoder of the antigen prediction model, two-time convolution on the β chain genetic information of the T cell receptor to obtain β chain genetic features of the T cell receptor. The α chain genetic features and the β chain genetic features of the T cell receptor constitute the genetic features of the T cell receptor.

Example 3: The server encodes, by the gene encoder of the antigen prediction model, the VJ information of the α chain and the VDJ information of the β chain of the immune cell receptor to obtain genetic features of the immune cell receptor, and the genetic features of the immune cell receptor include α chain genetic features and β chain genetic features of the immune cell receptor.

In some embodiments, the antigen prediction model includes two gene encoders. The server concatenates, by the first gene encoder of the antigen prediction model, the VJ information of the α chain of the T cell receptor to obtain α chain genetic information of the T cell receptor. The server concatenates, by the second gene encoder of the antigen prediction model, the VDJ information of the β chain of the T cell receptor to obtain β chain genetic information of the T cell receptor. The server encodes, by the first gene encoder of the antigen prediction model, the α chain genetic information of the T cell receptor based on the attention mechanism to obtain α chain genetic features of the T cell receptor. The server encodes, by the second gene encoder of the antigen prediction model, the β chain genetic information of the T cell receptor based on the attention mechanism to obtain β chain genetic features of the T cell receptor. The α chain genetic features and the β chain genetic features of the T cell receptor constitute the genetic features of the T cell receptor.

Second part: The server encodes, by the sequence encoder of the antigen prediction model, the amino acid sequence of the immune cell receptor to obtain sequence features of the immune cell receptor.

In some embodiments, in a case that the immune cell receptor is a B-cell receptor, the server encodes, by the sequence encoder of the antigen prediction model, amino acid sequences of the light chain and the heavy chain of the immune cell receptor based on the attention mechanism to obtain the sequence features of the immune cell receptor, and the sequence features of the immune cell receptor include light chain sequence features and heavy chain sequence features of the immune cell receptor. In some embodiments, the sequence encoder is an encoder of a Transformer model.

For example, the antigen prediction model includes two sequence encoders. In a case that the immune cell receptor is the B-cell receptor, the server performs, by the first sequence encoder of the antigen prediction model, embedded encoding on the amino acid sequence of the light chain of the B-cell receptor to obtain light chain embedding features of the B-cell receptor. A light chain embedding feature corresponds to an amino acid on the light chain, that is, a light chain embedding feature represents the amino acid embedding feature of an amino acid on the light chain. The server encodes, by the first sequence encoder, the plurality of light chain embedding features based on the sequence of a plurality of amino acids in the amino acid sequence of the B-cell receptor to obtain attention weights of the light chain embedding features. The server performs, by the first sequence encoder, weight fusion on the plurality of light chain embedding features based on the attention weights of the plurality of light chain embedding features to obtain the light chain sequence features of the B-cell receptor. The server performs, by the second sequence encoder of the antigen prediction model, embedded encoding on the amino acid sequence of the heavy chain of the B-cell receptor to obtain heavy chain embedding features of the B-cell receptor. A heavy chain embedding feature corresponds to an amino acid on the heavy chain, that is, a heavy chain embedding feature represents the amino acid embedding feature of an amino acid on the heavy chain. The server encodes, by the second sequence encoder, the plurality of heavy chain embedding features based on the sequence of a plurality of amino acids in the amino acid sequence of the B-cell receptor to obtain attention weights of the heavy chain embedding features. The server performs, by the second sequence encoder, weight fusion on the plurality of heavy chain embedding features based on the attention weights of the plurality of heavy chain embedding features to obtain the heavy chain sequence features of the B-cell receptor. The light chain sequence features and the heavy chain sequence features of the B-cell receptor constitute the sequence features of the B-cell receptor. In some embodiments, embedded encoding is implemented by a one-hot method or other methods, which is not limited herein.

In some embodiments, in a case that the immune cell receptor is a T cell receptor, the server encodes, by the sequence encoder of the antigen prediction model, amino acid sequences of the α chain and the β chain of the immune cell receptor based on the attention mechanism to obtain the sequence features of the immune cell receptor, and the sequence features of the immune cell receptor include α chain sequence features and β chain sequence features of the immune cell receptor.

For example, the antigen prediction model includes two sequence encoders. In a case that the immune cell receptor is the B-cell receptor, the server performs, by the first sequence encoder of the antigen prediction model, embedded encoding on the amino acid sequence of the α chain of the T cell receptor to obtain α chain embedding features of the T cell receptor. An α chain embedding feature corresponds to an amino acid on the α chain, that is, an α chain embedding feature represents the amino acid embedding feature of an amino acid on the α chain. The server encodes, by the first sequence encoder, the plurality of α chain embedding features based on the sequence of a plurality of amino acids in the amino acid sequence of the T cell receptor to obtain attention weights of the α chain embedding features. The server performs, by the first sequence encoder, weight fusion on the plurality of α chain embedding features based on the attention weights of the plurality of α chain embedding features to obtain the α chain sequence features of the T cell receptor. The server performs, by the second sequence encoder of the antigen prediction model, embedded encoding on the amino acid sequence of the β chain of the T cell receptor to obtain β chain embedding features of the T cell receptor. A β chain embedding feature corresponds to an amino acid on the β chain, that is, a β chain embedding feature represents the amino acid embedding feature of an amino acid on the β chain. The server encodes, by the second sequence encoder, the plurality of β chain embedding features based on the sequence of a plurality of amino acids in the amino acid sequence of the T cell receptor to obtain attention weights of the β chain embedding features. The server performs, by the second sequence encoder, weight fusion on the plurality of β chain embedding features based on the attention weights of the plurality of β chain embedding features to obtain the β chain sequence features of the T cell receptor. The α chain sequence features and the β chain sequence features of the T cell receptor constitute the sequence features of the T cell receptor.

304. The server integrates, by the antigen prediction model, the genetic features, the sequence features, and the three-dimensional structure features of the immune cell receptor, thereby obtaining receptor features of the immune cell receptor.

The receptor features of the immune cell receptor are obtained by integrating the genetic features, the sequence features, and the three-dimensional structure features, allowing the representation of the immune cell receptor from three aspects including the gene, sequence and structure. The receptor features can comprehensively represent the immune cell receptor.

In some embodiments, the server concatenates, by a feature fusion module of the antigen prediction model, the genetic features and the sequence features of the immune cell receptor, thereby obtaining gene and sequence integrated features of the immune cell receptor. The server performs, by the feature fusion module of the antigen prediction model, weight fusion on the gene and sequence integrated features and the three-dimensional structure features of the immune cell receptor based on a gate control attention mechanism to obtain receptor features of the immune cell receptor.

In some embodiments, the server can first integrate the genetic features and the sequence features of the immune cell receptor through the feature fusion module, thereby obtaining the gene and sequence integrated features of the immune cell receptor. The server then integrates the gene and sequence integrated features and the three-dimensional structure features through the gate control attention mechanism, thereby finally obtaining the receptor features of the immune cell receptor. The introduction of the gate control attention mechanism allows the model to pay more attention to the content with higher importance. Through the feature fusion method provided by the above implementation, the genetic features, the sequence features, and the three-dimensional structure features can be organically combined, and the obtained receptor features have a higher ability of expression.

In a case that the immune cell receptor is a B-cell receptor, the genetic features of the B-cell receptor include light chain genetic features and heavy chain genetic features of the B-cell receptor, and the sequence features of the B-cell receptor include light chain sequence features and heavy chain sequence features of the B-cell receptor. The server adds, by the feature fusion module, the light chain genetic features and the light chain sequence features of the B-cell receptor to obtain light chain gene and sequence features of the B-cell receptor. The server adds, by the feature fusion module, the heavy chain genetic features and the heavy chain sequence features of the B-cell receptor to obtain heavy chain gene and sequence features of the B-cell receptor. The server concatenates, by the feature fusion module, the light chain gene and sequence features and the heavy chain gene and sequence features of the B-cell receptor to obtain gene and sequence integrated features of the B-cell receptor. The server encodes, by the feature fusion module, the gene and sequence integrated features and the three-dimensional structure features of the B-cell receptor based on the attention mechanism, thereby obtaining a first attention weight for encoding the three-dimensional structure features with the gene and sequence integrated features, as well as a second attention weight for encoding the gene and sequence integrated features with the three-dimensional structure features. The server processes, by the feature fusion module, the first attention weight and the second attention weight based on a gate function, thereby obtaining a first gate weight and a second gate weight. The first gate weight and the second gate weight are used for controlling flow of information during feature fusion. The server performs, by the feature fusion module, weight fusion on the gene and sequence integrated features and the three-dimensional structure features of the B-cell receptor based on the first gate weight, thereby obtaining target gene and sequence integrated features of the B-cell receptor. In some embodiments, the target gene and sequence integrated features are obtained by multiplying the first gate weight with the three-dimensional structure features and adding the product to the gene and sequence integrated features. The server performs, by the feature fusion module, weight fusion on the gene and sequence integrated features and the three-dimensional structure features of the B-cell receptor based on the second gate weight, thereby obtaining target three-dimensional structure features of the B-cell receptor. In some embodiments, the target three-dimensional structure features are obtained by multiplying the second gate weight with the gene and sequence integrated features and adding the product to the three-dimensional structure features. The server performs, by the feature fusion module, tensor fusion on the target gene and sequence integrated features and the target three-dimensional structure features, such as multiplying the target gene and sequence integrated features with the target three-dimensional structure features, thereby obtaining initial receptor features of the B-cell receptor. The server performs, by the feature fusion module, at least two times of full connection on the initial receptor features of the B-cell receptor, thereby obtaining receptor features of the B-cell receptor.

In a case that the immune cell receptor is a T cell receptor, the genetic features of the T cell receptor include α chain genetic features and β chain genetic features of the T cell receptor, and the sequence features of the T cell receptor include α chain sequence features and β chain sequence features of the T cell receptor. The server adds, by the feature fusion module, the α chain genetic features and the α chain sequence features of the T cell receptor to obtain α chain gene and sequence features of the T cell receptor. The server adds, by the feature fusion module, the β chain genetic features and the β chain sequence features of the T cell receptor to obtain β chain gene and sequence features of the T cell receptor. The server concatenates, by the feature fusion module, the α chain gene and sequence features and the β chain gene and sequence features of the T cell receptor to obtain gene and sequence integrated features of the T cell receptor. The server encodes, by the feature fusion module, the gene and sequence integrated features and the three-dimensional structure features of the T cell receptor based on the attention mechanism, thereby obtaining a third attention weight for encoding the three-dimensional structure features with the gene and sequence integrated features, as well as a fourth attention weight for encoding the gene and sequence integrated features with the three-dimensional structure features. The server processes, by the feature fusion module, the third attention weight and the fourth attention weight based on the gate function, thereby obtaining a third gate weight and a fourth gate weight. The third gate weight and the fourth gate weight are used for controlling flow of information during feature fusion. The server performs, by the feature fusion module, weight fusion on the gene and sequence integrated features and the three-dimensional structure features of the T cell receptor based on the third gate weight, thereby obtaining target gene and sequence integrated features of the T cell receptor. In some embodiments, the target gene and sequence integrated features are obtained by multiplying the third gate weight with the three-dimensional structure features and adding the product to the gene and sequence integrated features. The server performs, by the feature fusion module, weight fusion on the gene and sequence integrated features and the three-dimensional structure features of the T cell receptor based on the fourth gate weight, thereby obtaining target three-dimensional structure features of the T cell receptor. In some embodiments, the target three-dimensional structure features are obtained by multiplying the fourth gate weight with the gene and sequence integrated features and adding the product to the three-dimensional structure features. The server performs, by the feature fusion module, tensor fusion on the target gene and sequence integrated features and the target three-dimensional structure features, such as multiplying the target gene and sequence integrated features with the target three-dimensional structure features, thereby obtaining initial receptor features of the T cell receptor. The server performs, by the feature fusion module, at least two times of full connection on the initial receptor features of the T cell receptor, thereby obtaining receptor features of the T cell receptor.

In some embodiments, the server adds, by the feature fusion module of the antigen prediction model, the genetic features and the sequence features of the immune cell receptor, thereby obtaining gene and sequence integrated features of the immune cell receptor. The server performs, by the feature fusion module, concatenation and at least one time of full connection on the gene and sequence integrated features and the three-dimensional structure features of the immune cell receptor to obtain receptor features of the immune cell receptor.

In some embodiments, the server can rapidly integrate, by the feature fusion module, the genetic features, the sequence features, and the three-dimensional structure features of the immune cell receptor through manners of addition, concatenation, and full connection, thereby obtaining receptor features of the immune cell receptor. The extraction efficiency of the receptor features is high.

In a case that the immune cell receptor is a B-cell receptor, the genetic features of the B-cell receptor include light chain genetic features and heavy chain genetic features of the B-cell receptor, and the sequence features of the B-cell receptor include light chain sequence features and heavy chain sequence features of the B-cell receptor. The server adds, by the feature fusion module, the light chain genetic features and the light chain sequence features of the B-cell receptor to obtain light chain gene and sequence features of the B-cell receptor. The server adds, by the feature fusion module, the heavy chain genetic features and the heavy chain sequence features of the B-cell receptor to obtain heavy chain gene and sequence features of the B-cell receptor. The light chain gene and sequence features and the heavy chain gene and sequence features of the B-cell receptor constitute gene and sequence integrated features of the B-cell receptor. The server performs, by the feature fusion module, concatenation on the gene and sequence integrated features and the three-dimensional structure features of the B-cell receptor to obtain initial receptor features of the B-cell receptor. The server performs, by the feature fusion module, at least one time of full connection on the initial receptor features of the B-cell receptor, thereby obtaining receptor features of the B-cell receptor.

In a case that the immune cell receptor is a T cell receptor, the genetic features of the T cell receptor include α chain genetic features and β chain genetic features of the T cell receptor, and the sequence features of the T cell receptor include α chain sequence features and β chain sequence features of the T cell receptor. The server adds, by the feature fusion module, the α chain genetic features and the α chain sequence features of the T cell receptor to obtain α chain gene and sequence features of the T cell receptor. The server adds, by the feature fusion module, the β chain genetic features and the β chain sequence features of the T cell receptor to obtain β chain gene and sequence features of the T cell receptor. The α chain gene and sequence features and the β chain gene and sequence features of the T cell receptor constitute gene and sequence integrated features of the T cell receptor. The server performs, by the feature fusion module, concatenation on the gene and sequence integrated features and the three-dimensional structure features of the T cell receptor to obtain initial receptor features of the T cell receptor. The server performs, by the feature fusion module, at least one time of full connection on the initial receptor features of the T cell receptor, thereby obtaining receptor features of the T cell receptor.

The above description illustrates an example where the server integrates the genetic features, the sequence features, and the three-dimensional structure features of the immune cell receptor to obtain the receptor features of the immune cell receptor. In some embodiments, in addition to integrating the genetic features, the sequence features, and the three-dimensional structure features of the immune cell receptor, the server may also integrate other information to obtain the receptor features of the immune cell receptor, and reference is made to the following implementation.

In some embodiments, the server integrates, by the feature fusion module of the antigen prediction model, the genetic features, the sequence features, and the three-dimensional structure features of the immune cell receptor, and physicochemical information of the amino acids in the immune cell receptor to obtain receptor features of the immune cell receptor.

The physicochemical information of the amino acids in the immune cell receptor includes physical properties and chemical properties of the amino acids. The physical properties include a basic composition and structure, solubility, a melting point, a boiling point, an optical behavior, optical rotation, etc. The chemical properties include acidity, basicity, hydrophobicity, etc. Introducing the physicochemical information of the amino acids to the receptor features of the immune cell receptor can enhance the expression capability of the receptor features, thereby achieving a more comprehensive representation of the immune cell receptor by the receptor features.

For example, the server concatenates, by the feature fusion module, the genetic features and the sequence features of the immune cell receptor, thereby obtaining gene and sequence integrated features of the immune cell receptor. The server performs, by the feature fusion module of the antigen prediction model, weight fusion on the gene and sequence integrated features and the three-dimensional structure features of the immune cell receptor based on the gate control attention mechanism to obtain initial receptor features of the immune cell receptor. The server adds, by the feature fusion module, the initial receptor features of the immune cell receptor and the physicochemical information of the amino acids in the immune cell receptor to obtain receptor features of the immune cell receptor.

305. The server performs, by the antigen prediction model, full connection and normalization on the receptor features of the immune cell receptor to output probabilities of the immune cell receptor corresponding to a plurality of candidate antigens.

In some embodiments, the server performs, by a classification module of the antigen prediction model, full connection on the receptor features of the immune cell receptor to obtain a classification matrix for the immune cell receptor. The server performs, by the classification module, normalization on the classification matrix of the immune cell receptor to obtain a probability set corresponding to the immune cell receptor. The probability set includes a plurality of probabilities, and each probability corresponds to a candidate antigen. The classification module is also referred to as a classification head.

In the above process, the server is pre-configured with the plurality of candidate antigens. The antigen prediction model performs the full connection and normalization on the receptor features of the immune cell receptor and outputs the probability of the immune cell receptor being associated with each candidate antigen. The probability represents the likelihood of the immune cell receptor being associated with the candidate antigen, or, the probability represents the likelihood of expected specific binding between the candidate antigen and the immune cell receptor.

306. The server determines a target antigen from the plurality of candidate antigens based on the probabilities of the immune cell receptor corresponding to the plurality of candidate antigens.

In some embodiments, the server determines, by the classification module, the candidate antigen corresponding to the probability in the probability set that meets target conditions as the target antigen. The probability set includes a plurality of probabilities, and each probability corresponds to a candidate antigen. In some embodiments, the probability that meets the target conditions refers to the highest probability in the probability set or the probability in the probability set that is greater than or equal to a probability threshold. The probability threshold is set by technical personnel according to actual situations, which is not limited herein. In some embodiments, the classification module includes a multilayer perceptron (MLP).

In the above process, the server determines, based on the probability of the immune cell receptor being associated with each candidate antigen, the antigen that can specifically bind to the immune cell receptor from the plurality of candidate antigens. Therefore, the antigen that can specifically bind to the immune cell receptor can be screened from the plurality of candidate antigens, facilitating guiding subsequent scientific research or vaccine design.

In some embodiments, the server performs, by the classification module of the antigen prediction model, prediction based on the receptor features, ultimately obtaining the target antigen corresponding to the immune cell receptor. There is no need for repetitive experiments, resulting in higher efficiency.

Operations 301 to 306 are described in conjunction with FIG. 5 as below.

Referring to FIG. 5, the server inputs genetic information, sequence information, and three-dimensional structure information of the immune cell receptor into an antigen prediction model. The antigen prediction model includes a gene encoder 501, a sequence encoder 502, and a structure encoder 503. The server encodes, by the gene encoder 501, the genetic information of the immune cell receptor to obtain genetic features of the immune cell receptor. The server encodes, by the sequence encoder 502, the sequence information of the immune cell receptor to obtain sequence features of the immune cell receptor. The server encodes, by the structure encoder 503, the three-dimensional structure information of the immune cell receptor to obtain three-dimensional structure features of the immune cell receptor. The antigen prediction model further includes a feature fusion module 504. The server concatenates, by the feature fusion module 504, the genetic features and the sequence features of the immune cell receptor, thereby obtaining gene and sequence integrated features h_{bio} of the immune cell receptor. The server performs, by the feature fusion module of the antigen prediction model, weight fusion on the gene and sequence integrated features h_{bio} and the three-dimensional structure features hₛₜᵣᵤ of the immune cell receptor based on the gate control attention mechanism to obtain target gene and sequence integrated features h^{/}_{bio} and target three-dimensional structure features h^{/}ₛₜᵣᵤ of the immune cell receptor. The server multiplies, by the feature fusion module 504, the target gene and sequence integrated features h^{/}_{bio} and the target three-dimensional structure features h^{/}ₛₜᵣᵤ to obtain initial receptor features h_{fusion} of the B-cell receptor. The server performs, by the feature fusion module 504, two times of full connection (FC1 and FC2) on the initial receptor features h_{fusion}, thereby obtaining receptor features Representation of the B-cell receptor. The antigen prediction model further includes a classification module. The server performs, by the classification module of the antigen prediction model, antigen prediction based on the receptor features of the immune cell receptor, and determines a target antigen 505 corresponding to the immune cell receptor from the plurality of candidate antigens. The target antigen 505 refers to an antigen that can specifically bind to the immune cell receptor.

The above description process is performed by the example of the server performing operations 301 to 306. In some embodiments, operations 301 to 306 are performed by a terminal. Both the terminal and the server serve as examples of a computer device, which is not limited herein.

Some embodiments may be formed by using any combination of all the foregoing technical solutions, and details are not described herein.

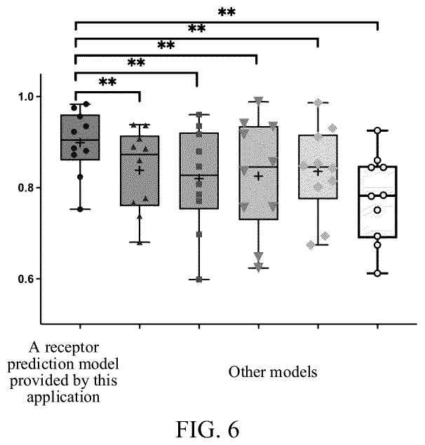

FIG. 6 illustrates results of testing on a public dataset by an antigen prediction method according to some embodiments. Referring to FIG. 6, which provides an accuracy rate of testing on the public dataset by an antigen prediction model provided in some embodiments. From FIG. 6, it can be observed that the antigen prediction model provided in some embodiments has a higher accuracy rate compared to other models in related art.

Through the technical solution provided by some embodiments, the antigen prediction model performs the feature extraction on the genetic information and sequence of the immune cell receptor to obtain the genetic features and the sequence features of the immune cell receptor. In the process of obtaining the receptor features of the immune cell receptor, the genetic features, the sequence features, and the three-dimensional structure features are integrated. The introduction of the three-dimensional structure features enriches the content of the receptor features and enhances the expression capability of the receptor features. Therefore, when antigen prediction is performed based on the receptor features, the accuracy of the obtained target antigen is high.

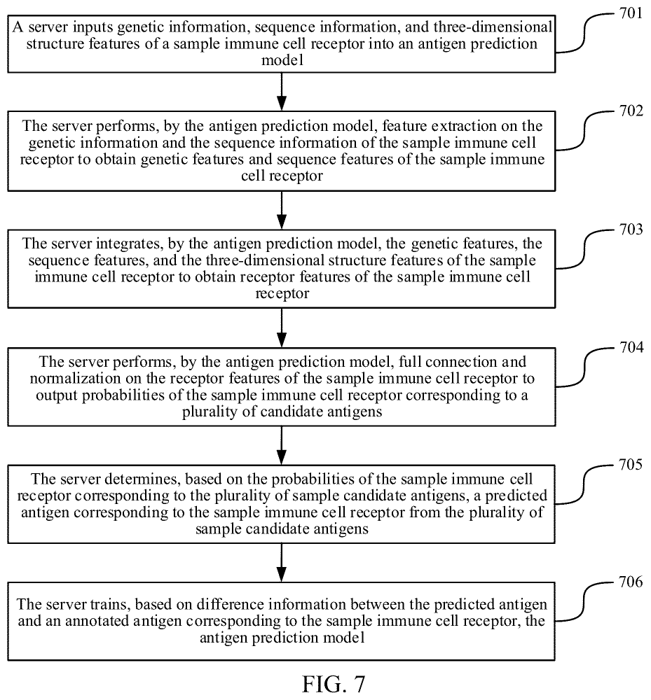

In order to provide a clearer description of the antigen prediction method provided by some embodiments. A method for training the antigen prediction model provided by some embodiments is described. Referring to FIG. 7, the method is executed primarily by a computer device, and taking the computer device being a server as an example, the method includes the following operations.

701. The server inputs genetic information, sequence information, and three-dimensional structure features of a sample immune cell receptor into the antigen prediction model.

Operation 701 and operation 302 belong to the same inventive concept, and for the implementation process, reference is made to related descriptions in operation 302, which will not be repeated herein.

702. The server performs feature extraction on the genetic information and the sequence information of the sample immune cell receptor through the antigen prediction model to obtain genetic features and sequence features of the sample immune cell receptor.

Operation 702 and operation 303 belong to the same inventive concept, and for the implementation process, reference is made to related descriptions in operation 303, which will not be repeated herein.

703. The server integrates, by the antigen prediction model, the genetic features, the sequence features, and the three-dimensional structure features of the sample immune cell receptor, thereby obtaining receptor features of the sample immune cell receptor.

Operation 703 and operation 304 belong to the same inventive concept, and for the implementation process, reference is made to related descriptions in operation 304, which will not be repeated herein.

704. The server performs, by the antigen prediction model, full connection and normalization on the receptor features of the sample immune cell receptor to output probabilities of the sample immune cell receptor corresponding to a plurality of candidate antigens.

In other words, the antigen prediction model performs the full connection and normalization on the receptor features of the sample immune cell receptor and outputs the probability of the sample immune cell receptor being associated with each sample candidate antigen. The probability represents the likelihood of the sample immune cell receptor being associated with the sample candidate antigen, or, the probability represents the expected likelihood of specific binding between the sample candidate antigen and the sample immune cell receptor.

Operation 704 and operation 305 belong to the same inventive concept, and for the implementation process, reference is made to related descriptions in operation 305, which will not be repeated herein.

705. The server determines, based on the probabilities of the sample immune cell receptor corresponding to the plurality of sample candidate antigens, a predicted antigen corresponding to the sample immune cell receptor from the plurality of sample candidate antigens.

In other words, based on the probabilities of the sample immune cell receptor being associated with each sample candidate antigen, the predicted antigen of the sample immune cell receptor is determined from the plurality of sample candidate antigens, that is, the sample candidate antigen indicated by the probability meeting a target condition is adopted as the predicted antigen of the sample immune cell receptor.

Operation 705 and operation 306 belong to the same inventive concept, and for the implementation process, reference is made to related descriptions in operation 306, which will not be repeated herein.

706. The server trains, based on difference information between the predicted antigen and an annotated antigen corresponding to the sample immune cell receptor, the antigen prediction model.

In other words, the antigen prediction model is trained based on the difference information between the predicted antigen and the annotated antigen of the sample immune cell receptor. The annotated antigen refers to an antigen that can specifically bind to the sample immune cell receptor.

In some embodiments, the server constructs a cross-entropy loss function based on the difference information between the predicted antigen and the annotated antigen corresponding to the immune cell receptor. The server trains, by a gradient descent algorithm, the antigen prediction model based on the cross-entropy loss function, that is, model parameters of the antigen prediction model are adjusted.

Operations 701 to 706 are described by the example of one round of training of the antigen prediction model by the server. The process of performing multiple rounds of training on the antigen prediction model and operations 701 to 706 belong to the same inventive concept, which are not repeated herein.

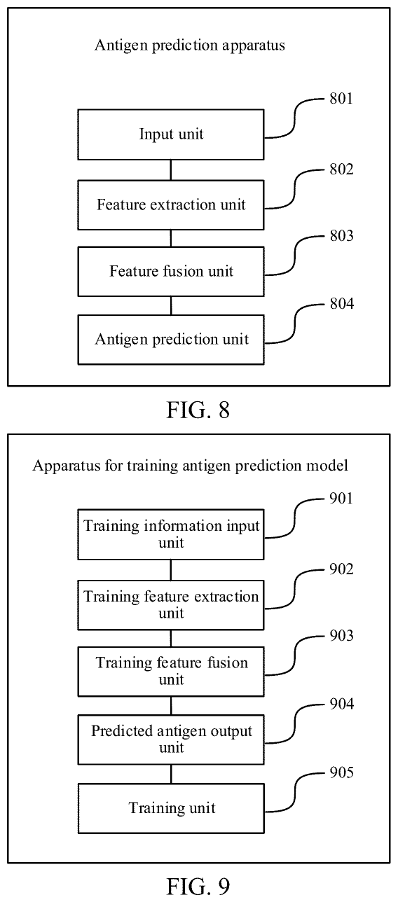

FIG. 8 is a schematic structural diagram of an antigen prediction apparatus according to some embodiments. Referring to FIG. 8, the apparatus includes: an input unit 801, a feature extraction unit 802, a feature fusion unit 803, and an antigen prediction unit 804.

The input unit 801 is configured to input genetic information, sequence information, and three-dimensional structure features of an immune cell receptor into an antigen prediction model.

The feature extraction unit 802 is configured to perform, through the antigen prediction model, feature extraction on the genetic information and the sequence information of the immune cell receptor to obtain genetic features and sequence features of the immune cell receptor.

The feature fusion unit 803 is configured to perform, by the antigen prediction model, the genetic features, the sequence features, and the three-dimensional structure features of the immune cell receptor to obtain receptor features of the immune cell receptor.

The antigen prediction unit 804 is configured to perform, by the antigen prediction model, full connection and normalization on the receptor features of the immune cell receptor to output probabilities of the immune cell receptor being associated with each candidate antigen; and determine, based on the probabilities of the immune cell receptor being associated with each candidate antigen, an antigen specifically binding to the immune cell receptor from the plurality of candidate antigens.

In some embodiments, in a case that the genetic information includes VDJ information of the immune cell receptor, the feature extraction unit 802 encodes, by a gene encoder of the antigen prediction model, the VDJ information of the immune cell receptor to obtain genetic features of the immune cell receptor, where V represents an encoding variable region, D represents an encoding hypervariable region, and J represents an encoding joining region.

In some embodiments, in a case that the sequence information includes an amino acid sequence of the immune cell receptor, the feature extraction unit 802 encodes, by a sequence encoder of the antigen prediction model, the amino acid sequence of the immune cell receptor to obtain sequence features of the immune cell receptor.

In some embodiments, the feature extraction unit 802 is configured to perform any one of the following:

in a case that the immune cell receptor is a B-cell receptor, VJ information of light chains and VDJ information of heavy chains of the immune cell receptor are encoded to obtain genetic features of the immune cell; and

in a case that the immune cell receptor is a T cell receptor, VJ information of an α chain and VDJ information of a β chain of the immune cell receptor are encoded to obtain genetic features of the immune cell receptor.

In some embodiments, the feature extraction unit 802 is configured to perform full connection on the VJ information of the light chains and the VDJ information of the heavy chains of the immune cell receptor to obtain genetic features of the immune cell receptor, where the genetic features of the immune cell receptor include light chain genetic features and heavy chain genetic features of the immune cell receptor; or, in some embodiments, perform full connection on the VJ information of the α chain and the VDJ information of the β chain of the immune cell receptor to obtain genetic features of the immune cell receptor, where the genetic features of the immune cell receptor include α chain genetic features and β chain genetic features of the immune cell receptor.

In some embodiments, the feature extraction unit 802 is configured to perform any one of the following:

in a case that the immune cell receptor is a B-cell receptor, the sequence encoder of the antigen prediction model encodes amino acid sequences of the light chains and the heavy chains of the immune cell receptor based on the attention mechanism to obtain sequence features of the immune cell receptor, where the sequence features of the immune cell receptor include light chain sequence features and heavy chain sequence features of the immune cell receptor; and

in a case that the immune cell receptor is a T cell receptor, the sequence encoder of the antigen prediction model encodes amino acid sequences of the α chain and the β chain of the immune cell receptor based on the attention mechanism to obtain sequence features of the immune cell receptor, where the sequence features of the immune cell receptor include α chain sequence features and β chain sequence features of the immune cell receptor.

In some embodiments, the feature fusion unit 803 is configured to concatenate, by a feature fusion module of the antigen prediction model, the genetic features and the sequence features of the immune cell receptor to obtain gene and sequence integrated features of the immune cell receptor; and perform, based on the gate control attention mechanism, weight fusion on the gene and sequence integrated features and the three-dimensional structure features of the immune cell receptor to obtain receptor features of the immune cell receptor.

In some embodiments, the apparatus further includes:

a three-dimensional structure feature obtaining unit, configured to obtain an amino acid sequence containing a CDR3 region of the immune cell receptor; performing multiple sequence alignment on the amino acid sequence to obtain at least one reference amino acid sequence, where the similarity between the reference amino acid sequence and the amino acid sequence satisfies a similarity condition; obtaining a homologous template of the amino acid sequence, where the homologous template includes structural information of homologous sequences of the amino acid sequence; and performing multiple rounds of iterations based on the amino acid sequence, at least one reference amino acid sequence, and the homologous template to obtain three-dimensional structure features of the immune cell receptor.

In some embodiments, the apparatus further includes:

a three-dimensional structure feature obtaining unit, configured to obtain three-dimensional structure information of the immune cell receptor, where the three-dimensional structure information includes three-dimensional coordinates of a plurality of amino acids in the immune cell receptor; and performing graph convolution on the three-dimensional structure information of the immune cell receptor to obtain three-dimensional structure features of the immune cell receptor, or encode, based on the attention mechanism, the three-dimensional structure information of the immune cell receptor to obtain three-dimensional structure features of the immune cell receptor.

In some embodiments, the feature fusion unit 803 is further configured to integrate, by the antigen prediction model, the genetic features, the sequence features, and the three-dimensional structure features of the immune cell receptor, and physicochemical information of the amino acids in the immune cell receptor to obtain receptor features of the immune cell receptor.

When the antigen prediction apparatus provided in the foregoing embodiment performs antigen prediction, division of the foregoing functional modules is merely used as an example for description. In some embodiments, the above functions may be allocated to different functional modules to be completed according to requirements. That is, an internal structure of a computer device is divided into different functional modules, to complete all or some of the functions described above. In addition, the embodiments of the antigen prediction apparatus and the antigen prediction method provided in the foregoing embodiments belong to the same concept. For details of a specific implementation process, refer to the method embodiments. Details are not described herein again.

In some embodiments, the antigen prediction model performs the feature extraction on the genetic information and sequence of the immune cell receptor to obtain the genetic features and the sequence features of the immune cell receptor. In the process of obtaining the receptor features of the immune cell receptor, the genetic features, the sequence features, and the three-dimensional structure features are integrated. The introduction of the three-dimensional structure features enriches the content of the receptor features and enhances the expression capability of the receptor features. Therefore, when antigen prediction is performed based on the receptor features, the accuracy of the obtained target antigen is high.

FIG. 9 is a schematic structural diagram of an apparatus for training an antigen prediction model according to some embodiments. Referring to FIG. 9, the apparatus includes: a training information input unit 901, a training feature extraction unit 902, a training feature fusion unit 903, a predicted antigen output unit 904, and a training unit 905.

The training information input unit 901 is configured to input genetic information, sequence information, and three-dimensional structure features of a sample immune cell receptor into the antigen prediction model.

The training feature extraction unit 902 is configured to perform, by the antigen prediction model, feature extraction on the genetic information and the sequence information of the sample immune cell receptor to obtain genetic features and sequence features of the sample immune cell receptor.

The training feature fusion unit 903 is configured to integrate, by the antigen prediction model, the genetic features, the sequence features, and the three-dimensional structure features of the sample immune cell receptor to obtain receptor features of the sample immune cell receptor.

The predicted antigen output unit 904 is configured to perform, by the antigen prediction model, full connection and normalization on the receptor features of the sample immune cell receptor to output probabilities of the sample immune cell receptor being associated with each sample candidate antigen. Based on the probabilities of the sample immune cell receptor being associated with each sample candidate antigen, a predicted antigen of the sample immune cell receptor is determined from the plurality of sample candidate antigens.

The training unit 905 is configured to train the antigen prediction model based on difference information between the predicted antigen of the sample immune cell receptor and an annotated antigen of the sample immune cell receptor. The annotated antigen refers to an antigen that can specifically bind to the sample immune cell receptor.

When the apparatus for training the antigen prediction model provided in the some embodiments trains the antigen prediction model, division of the foregoing functional modules is merely used as an example for description. In some embodiments, the above functions are allocated to different functional modules to be completed according to requirements. That is, an internal structure of a computer device is divided into different functional modules, to complete all or some of the functions described above. In addition, the embodiments of the antigen prediction apparatus and the antigen prediction method provided in the foregoing embodiments belong to the same concept. For details of a specific implementation process, refer to the method embodiments. Details are not described herein again.

A person skilled in the art would understand that the above “units” could be implemented by hardware logic, a processor or processors executing computer software code, or a combination of both. The “units” may also be implemented in software stored in a memory of a computer or a non-transitory computer-readable medium, where the instructions of each unit are executable by a processor to thereby cause the processor to perform the respective operations of the corresponding unit

Additionally, the units may be separately or wholly combined into one or several other units, or one (or more) of the units may further be divided into a plurality of units of smaller functions. In this way, same operations may be implemented, and the implementation of the technical effects is not affected. The foregoing units are divided based on logical functions. In some embodiments, a function of one unit may also be implemented by multiple units, or functions of multiple units are implemented by one unit. In some embodiments, the apparatus may also include other units. In some embodiments, the functions may also be cooperatively implemented by other units and may be cooperatively implemented by a plurality of units

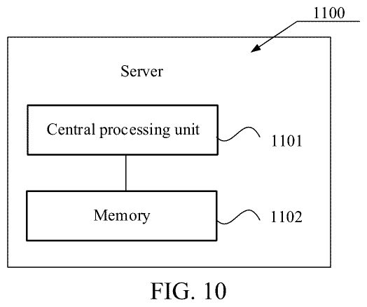

Some embodiments provide a computer device configured to perform the above method. The computer device is implemented as a terminal or a server. The server is used as an example below to introduce the structure of the server. FIG. 10 is a schematic structural diagram of a server according to some embodiments. The server 1100 may vary due to different configurations or performance. The server 1100 includes one or more central processing units (CPUs) 1101 and one or more memories 1102. The one or more memories 1102 store at least one computer program, and the at least one computer program is loaded and executed by the one or more central processing units 1101 to implement the antigen prediction method or the method for training an antigen prediction model provided in some method embodiments. Certainly, the server 1100 may be further provided with components such as a wired or wireless network interface, a keyboard, and an input/output interface, to facilitate input and output. The server 1100 may further include other components configured to implement device functions. Details are not further described herein.

In some embodiments, a computer-readable storage medium, such as a memory including a computer program, is further provided. The computer program may be executed by a central processing unit to complete the antigen prediction method or the method for training an antigen prediction model in the foregoing embodiments. The computer-readable storage medium herein may be a high-speed RAM memory, or a non-volatile memory, such as at least one disk memory. The computer-readable storage medium may also be a read-only memory (ROM), a random access memory (RAM), a compact disc read-only memory (CD-ROM), a magnetic tape, a floppy disk, an optical data storage device, etc.

In some embodiment, a computer program product or computer program is further provided. The computer program product or computer program includes program code. The program code is stored in the computer-readable storage medium. A central processing unit of a computer device reads the program code from the computer-readable storage medium. The central processing unit performs the program code, such that the computer device implements the antigen prediction method or the method for training an antigen prediction model.

In some embodiments, the computer program may be deployed on one computer device to be executed, or on a plurality of computer devices at one place to be executed, or on a plurality of computer devices distributed in several places and connected through a communication network. The plurality of computer devices distributed in the several places and connected through the communication network constitute a blockchain system.

A person of ordinary skill in the art understands that all or some of the operations of the foregoing are implemented by hardware or a program instructing related hardware. The program is stored in a computer-readable storage medium. The above-mentioned storage medium is a read-only memory, a magnetic disk, or an optical disc, or the like.

The foregoing embodiments are used for describing, instead of limiting the technical solutions of the disclosure. A person of ordinary skill in the art shall understand that although the disclosure has been described in detail with reference to the foregoing embodiments, modifications can be made to the technical solutions described in the foregoing embodiments, or equivalent replacements can be made to some technical features in the technical solutions, provided that such modifications or replacements do not cause the essence of corresponding technical solutions to depart from the spirit and scope of the technical solutions of the embodiments of the disclosure and the appended claims.

What is claimed is:

1. An antigen prediction method, executed by a computer device, comprising:
inputting genetic information, sequence information, and three-dimensional structure features of an immune cell receptor into an antigen prediction model;
performing, by the antigen prediction model, feature extraction on the genetic information and the sequence information to obtain genetic features and sequence features of the immune cell receptor;
integrating, by the antigen prediction model, the genetic features, the sequence features, and the three-dimensional structure features to obtain receptor features of the immune cell receptor;
performing, by the antigen prediction model, full connection and normalization on the receptor features to output a probability of the immune cell receptor being associated with each candidate antigen of a plurality of candidate antigens; and
determining, based on the probability of the immune cell receptor being associated with each candidate of the plurality of candidate antigens, an antigen binding to the immune cell receptor from the plurality of candidate antigens.

2. The antigen prediction method according to claim 1, the genetic information comprising VDJ information of the immune cell receptor, wherein the VDJ information includes V information that represents an encoding variable region, D information that represents an encoding hypervariable region, and J information that represents an encoding joining region; and
performing, by the antigen prediction model, the feature extraction on the genetic information to obtain the genetic features of the immune cell receptor comprises:
encoding, by a gene encoder of the antigen prediction model, the VDJ information to obtain the genetic features.

3. The antigen prediction method according to claim 1, wherein the sequence information comprises an amino acid sequence of the immune cell receptor; and
wherein performing, by the antigen prediction model, the feature extraction on the sequence information to obtain sequence features of the immune cell receptor comprises:
encoding, by a sequence encoder of the antigen prediction model, the amino acid sequence to obtain the sequence features.

4. The antigen prediction method according to claim 2, wherein encoding the VDJ information to obtain the genetic features comprises:
based on the immune cell receptor being a B-cell receptor, encoding V and J information of light chains and VDJ information of heavy chains of the immune cell receptor to obtain the genetic features; and
based on the immune cell receptor being a T cell receptor, encoding V and J information of an α chain and VDJ information of a β chain of the immune cell receptor to obtain the genetic features.

5. The antigen prediction method according to claim 4, wherein encoding the V and the J information of light chains and the VDJ information of heavy chains of the immune cell receptor to obtain the genetic features comprises:
performing full connection on the V and the J information of the light chains and the VDJ information of the heavy chains to obtain the genetic features, the genetic features comprising light chain genetic features and heavy chain genetic features of the immune cell receptor.

6. The antigen prediction method according to claim 4, wherein encoding the V and the J information of an α chain and the VDJ information of a β chain of the immune cell receptor to obtain the genetic features comprises:
performing full connection on the V and the J information of the α chain and the VDJ information of the β chain to obtain the genetic features, the genetic features comprising α chain genetic features and β chain genetic features of the immune cell receptor

7. The antigen prediction method according to claim 3, wherein encoding the amino acid sequence to obtain the sequence features comprises any one of the following:
based on the immune cell receptor being a B-cell receptor, encoding, based on an attention mechanism, amino acid sequences of light chains and heavy chains of the immune cell receptor to obtain the sequence features, the sequence features comprising light chain sequence features and heavy chain sequence features of the immune cell receptor; and
based on the immune cell receptor being a T cell receptor, encoding, based on the attention mechanism, amino acid sequences of an α chain and a β chain of the immune cell receptor to obtain the sequence features, the sequence features comprising α chain sequence features and β chain sequence features of the immune cell receptor.

8. The antigen prediction method according to claim 1, wherein integrating, by the antigen prediction model, the genetic features, the sequence features, and the three-dimensional structure features to obtain the receptor features of the immune cell receptor comprises:
concatenating, by a feature fusion module of the antigen prediction model, the genetic features and the sequence features to obtain gene and sequence integrated features of the immune cell receptor; and
performing, based on a gate control attention mechanism, weight fusion on the gene and sequence integrated features and the three-dimensional structure features to obtain the receptor features of the immune cell receptor.

9. The antigen prediction method according to claim 1, further comprising:
obtaining an amino acid sequence containing a CDR3 region of the immune cell receptor, the CDR3 region being a sub-region of a complementary determining region (CDR) that exists on the immune cell receptor;
performing multiple sequence alignment on the amino acid sequence to obtain at least one reference amino acid sequence, the similarity between the at least one reference amino acid sequence and the amino acid sequence satisfying a similarity condition;
obtaining a homologous template of the amino acid sequence, the homologous template comprising structural information of homologous sequences of the amino acid sequence; and
performing multiple rounds of iterations based on the amino acid sequence, the at least one reference amino acid sequence, and the homologous template to obtain the three-dimensional structure features.

10. The antigen prediction method according to claim 7, wherein the antigen prediction method further comprises any one of the following:
performing graph convolution on three-dimensional structure information of the immune cell receptor to obtain the three-dimensional structure features, the three-dimensional structure information comprising three-dimensional coordinates of a plurality of amino acids in the immune cell receptor; and
encoding, based on the attention mechanism, the three-dimensional structure information to obtain the three-dimensional structure features.

11. The antigen prediction method according to claim 10, wherein integrating, by the antigen prediction model, the genetic features, the sequence features, and the three-dimensional structure features to obtain the receptor features of the immune cell receptor comprises:
integrating, by the antigen prediction model, the genetic features, the sequence features, the three-dimensional structure features, and physicochemical information of the plurality of amino acids in the immune cell receptor to obtain the receptor features.

12. An antigen prediction apparatus comprising:
at least one memory configured to store program code; and
at least one processor configured to read the program code and operate as instructed by the program code, the program code comprising:
input code configured to cause at least one of the at least one processor to input genetic information, sequence information, and three-dimensional structure features of an immune cell receptor into an antigen prediction model;
feature extraction code configured to cause at least one of the at least one processor to perform, by the antigen prediction model, feature extraction on the genetic information and the sequence information to obtain genetic features and sequence features of the immune cell receptor;
feature fusion code configured to cause at least one of the at least one processor to integrate, by the antigen prediction model, the genetic features, the sequence features, and the three-dimensional structure features to obtain receptor features of the immune cell receptor; and
antigen prediction code configured to cause at least one of the at least one processor to perform, by the antigen prediction model, full connection and normalization on the receptor features to output a probability of the immune cell receptor being associated with each candidate antigen of a plurality of candidate antigens; and determining, based on the probability of the immune cell receptor being associated with each candidate of the plurality of candidate antigens, an antigen binding to the immune cell receptor from the plurality of candidate antigens.

13. The antigen prediction apparatus according to claim 12, the genetic information comprising VDJ information of the immune cell receptor, wherein the VDJ information includes V information that represents an encoding variable region, D information that represents an encoding hypervariable region, and J information that represents an encoding joining region; and
the feature extraction code is further configured to cause at least one of the at least one processor to:
encode, by a gene encoder of the antigen prediction model, the VDJ information to obtain the genetic features.

14. The antigen prediction apparatus according to claim 12, wherein the sequence information comprises an amino acid sequence of the immune cell receptor; and
wherein the feature extraction code is further configured to cause at least one of the at least one processor to:
encode, by a sequence encoder of the antigen prediction model, the amino acid sequence to obtain the sequence features.

15. The antigen prediction apparatus according to claim 13, wherein the feature extraction code is further configured to cause at least one of the at least one processor to:
based on the immune cell receptor being a B-cell receptor, encode V and J information of light chains and VDJ information of heavy chains of the immune cell receptor to obtain the genetic features; and
based on the immune cell receptor being a T cell receptor, encode V and J information of an α chain and VDJ information of a β chain of the immune cell receptor to obtain the genetic features.

16. The antigen prediction apparatus according to claim 15, wherein the feature extraction code is further configured to cause at least one of the at least one processor to:
perform full connection on the V and the J information of the light chains and the VDJ information of the heavy chains to obtain the genetic features, the genetic features comprising light chain genetic features and heavy chain genetic features of the immune cell receptor.

17. The antigen prediction apparatus according to claim 15, wherein the feature extraction code is further configured to cause at least one of the at least one processor to:
perform full connection on the V and the J information of the α chain and the VDJ information of the β chain to obtain the genetic features, the genetic features comprising α chain genetic features and β chain genetic features of the immune cell receptor

18. The antigen prediction apparatus according to claim 14, wherein the feature extraction code is further configured to cause at least one of the at least one processor to perform any one of the following:
based on the immune cell receptor being a B-cell receptor, encode, based on an attention mechanism, amino acid sequences of light chains and heavy chains of the immune cell receptor to obtain the sequence features, the sequence features comprising light chain sequence features and heavy chain sequence features of the immune cell receptor; and
based on the immune cell receptor being a T cell receptor, encode, based on the attention mechanism, amino acid sequences of an α chain and a β chain of the immune cell receptor to obtain the sequence features, the sequence features comprising α chain sequence features and β chain sequence features of the immune cell receptor.

19. The antigen prediction apparatus according to claim 12, wherein the feature fusion code is further configured to cause at least one of the at least one processor to:
concatenate, by a feature fusion module of the antigen prediction model, the genetic features and the sequence features to obtain gene and sequence integrated features of the immune cell receptor; and
perform, based on a gate control attention mechanism, weight fusion on the gene and sequence integrated features and the three-dimensional structure features to obtain the receptor features of the immune cell receptor.

20. A non-transitory computer-readable storage medium storing computer code which, when executed by at least one processor, causes the at least one processor to at least:
input genetic information, sequence information, and three-dimensional structure features of an immune cell receptor into an antigen prediction model;
perform, by the antigen prediction model, feature extraction on the genetic information and the sequence information to obtain genetic features and sequence features of the immune cell receptor;
integrate, by the antigen prediction model, the genetic features, the sequence features, and the three-dimensional structure features to obtain receptor features of the immune cell receptor;
perform, by the antigen prediction model, full connection and normalization on the receptor features to output a probability of the immune cell receptor being associated with each candidate antigen of a plurality of candidate antigens; and
determine, based on the probability of the immune cell receptor being associated with each candidate of the plurality of candidate antigens, an antigen binding to the immune cell receptor from the plurality of candidate antigens.
